(19) United States
(12) Plant Patent Application Publication
(10) Pub. No.: US 2013/0298298 P1
(43) Pub. Date: Nov. 7, 2013

(54) Beech mushroom plant named "FINC-1"

(50) ;

(71) Applicants: Jihong Cheng, (US); Yu Liu, (US)

(72) Inventors: Jihong Cheng, (US); Yu Liu, (US)

(21) Appl. No.: 13/506,635

(22) Filed: May 4, 2012

(51) Int. Cl. A01H 15/00

(52) U.S. Cl. PLT/394

(57) ABSTRACT

A new and distinct variety of mushroom plant of beech mushroom, Hypsizigus marmoreus, named “FINC-1”, is cultivated by gathering and repeated breeding of fungal strains with dominant traits to have short culture length, short growth length, high yield, good appearance, no bitterness, and it is exquisite in stability, reproducibility and uniformity when produced.

### BACKGROUND OF THE INVENTION

The new variety “FINC-1” of the invention is produced from two parental cultivars. One of the parental cultivars is “TNN-11” and the other cultivar is “Hakata-A264”, both of which were from Culture Collection of Shanghai Academy of Agricultural Sciences and were also commercially available from a supermarket in Japan.

The site of development was Shanghai Finc Bio-tech Inc. in Shanghai, China.

The parental cultivars were widely cultivated in Japan and China. The “TNN-11” has good morphological characters, i.e. uniform size cap, good-looking mottles and is not bitter, but it needs long culture length and long growth length, while “Hakata-A264” is superior in culture length and growth length, but is bitter.

Crossbreeding of “TNN-11” and “Hakata-A264” was performed to develop 400 new breed strains of mushrooms in August 2008. Then, cultivation experiments were carried out for verifying the trait characters of these breed strains from September 2008 to October 2009, consequently to chase up adequate fungal strains (named “TN4-52”) with short growth length, high yield, good appearance, and no bitterness.

Thereafter, further cultivation experiments were repeatedly carried out from November 2009 to August 2010 for corroborating stability, reproducibility and uniformity of the new strains under a small-scale number (2,300 bottles each time), and the result proved that the new fungal strains were fit for the expected purpose of the invention, thus it is named as “FINC-1” strains ultimately. The new fungal strain “FINC-1” has been registered as an edible mushroom strain on Sep. 16, 2010 by Shanghai Crop Variety Approval Committee. Then after that large-scale cultivation experiments (60,000 bottles each time) were repeatedly carried out from November 2010 to December 2011 for corroborating adaptability, and the result proved that “FINC-1” is suitable for large-scale production.

### SUMMARY OF THE INVENTION

The present invention is a new and distinct variety of mushroom characterized particularly by its short culture length, short growth length, high yield, good appearance, and no bitterness, and it is exquisite in stability, reproducibility and uniformity when being produced. This novel and distinct variety of mushroom is identified as “FINC-1”.

### BRIEF DESCRIPTION OF THE FIGURES

FIG. 1A shows a top view of the new variety of mushrooms named “FINC-1” of the present invention.

FIG. 1B shows a front view of the mushrooms of FIG. 1A.

FIG. 2 is a photograph showing a perspective view of the present plant variety cultivated in vases.

FIG. 3 shows the state of cultivating in bottles of a control variety “TNN-11” similar to the mushrooms of the invention.

FIG. 4 shows the state of cultivating in bottles of a control variety “Hakata-A264” similar to the mushrooms of the invention.

FIG. 5A and FIG. 5B are photographs showing a surface view and a back view, respectively, of the state of juxtapositional cultivation of the “Hakata-A264” mushrooms placed in juxtaposition. Similar to FIG. 5A and FIG. 5B, FIG. 5C and FIG. 5D are photographs of “Hokuto Shiro Ichigoukin” and FIG. 5E and FIG. 5F are photographs of “Hokuto Shiro Ichigoukin” and “Hakata-A264” of the state of juxtapositional cultivation. In these figures, the designation “4” is the abbreviation of “Hakata-A264” and “F-W”, “W” are the abbreviations of “Hokuto Shiro Ichigoukin”.

FIG. 6A and FIG. 6B are photographs showing a surface view and a back view, respectively, of the state of juxtapositional cultivation of the parent plants “TNN-11” (left), “Hakata-A264” (right) and the present variety (middle) mushroom placed in juxtaposition. In these figures, the designation “N” and “TNN” are the abbreviations of “TNN-11”, “N4” and “TN4-52-T1” are the abbreviations of “FINC-1”, “F-4” and “4” are the abbreviations of “Hakata-A264”.

FIG. 7A and FIG. 7B are photographs showing a surface view and a back view, respectively, of the state of juxtapositional cultivation of the parent plant “TNN-11” and the present variety of mushrooms placed in juxtaposition.

FIG. 8 is a photograph showing the differences between yield among “FINC-1” and the control variety of “TNN-11”.

FIG. 9 shows the state of “FINC-1” (yellowish, left) and “TNN-11” (brown, right) cultivated in the same room after fruit-body-formation promotion for 21 days.

### BOTANICAL DESCRIPTION

This invention relates to a new and distinct variety of mushroom plant of beech mushroom, Hypsizigus marmoreus, named “FINC-1”, which is cultivated by repeatedly breeding beech mushrooms with dominant traits. This new variety of mushroom has short culture length, short growth length, high yield, good appearance, no bitterness, and is exquisite in stability, reproducibility and uniformity when produced.

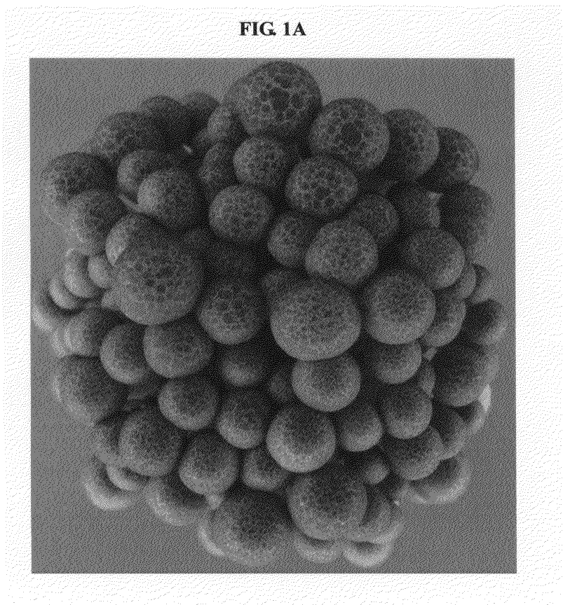

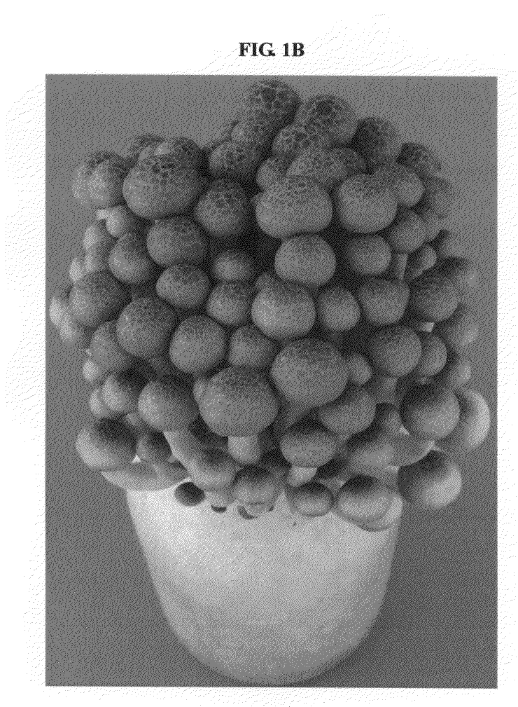

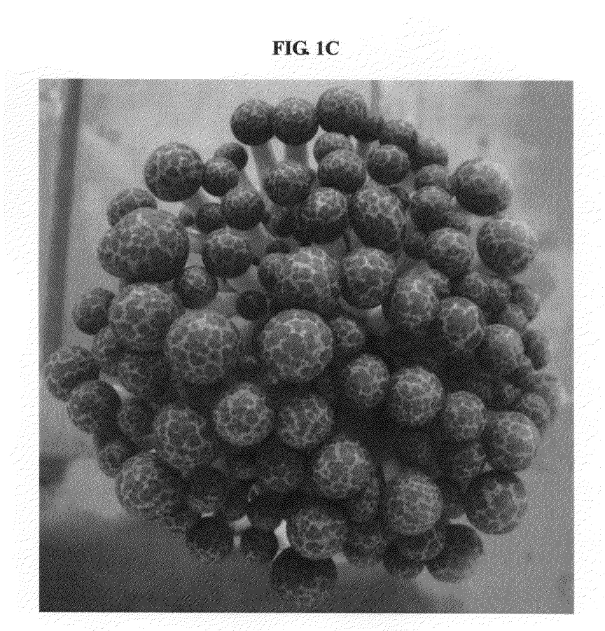

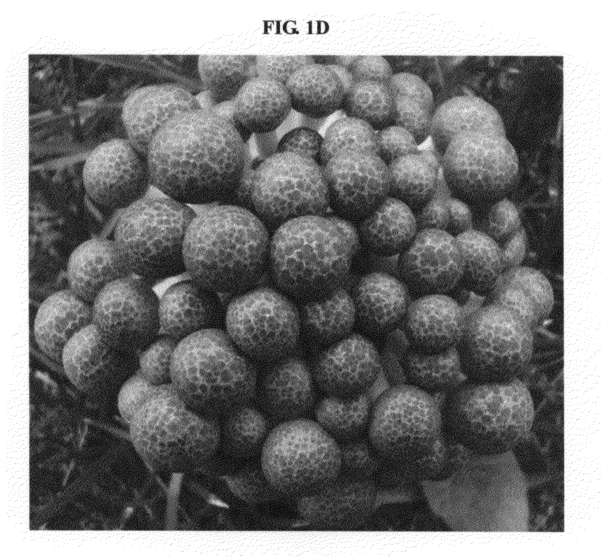

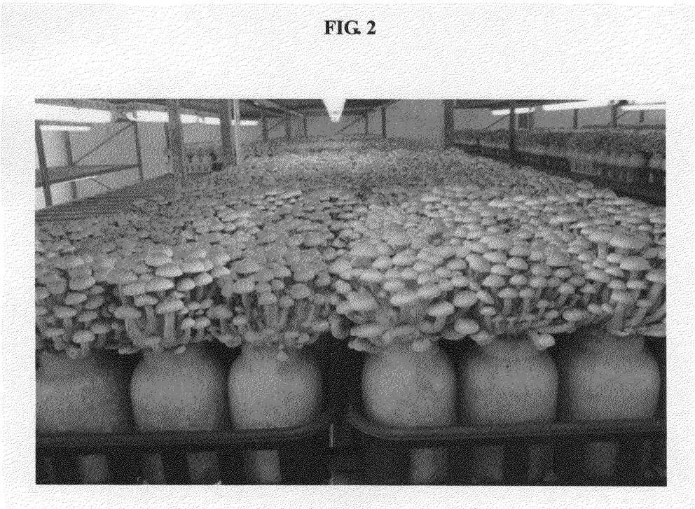

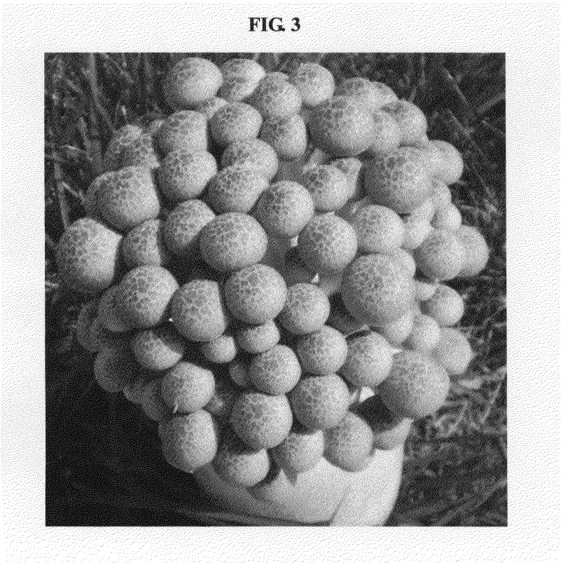

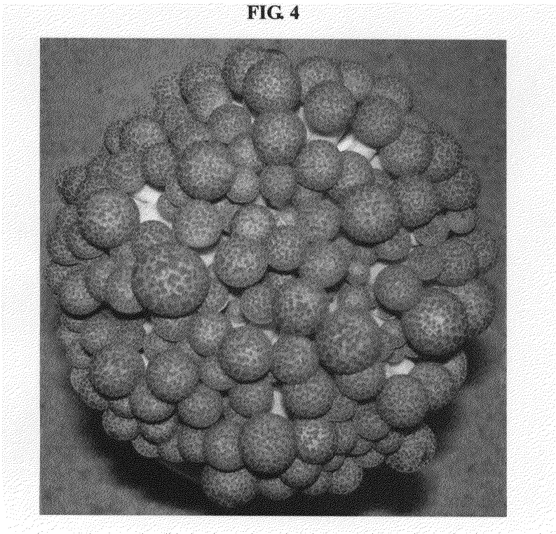

One actual sample of the new variety “FINC-1” of the invention is shown in FIGS. 1A and 1B. Needless to say, this illustrated sample should not be understood as one and only embodiment of the invention because plant morphology can be influenced not only by gene but also by culture medium, growth condition and so on. The morphological characteristics of the new variety “FINC-1” of the invention will be described in detail later.

The new variety “FINC-1” of the invention is produced from two parental cultivars. Both the parental cultivars were from Culture Collection of Shanghai Academy of Agricultural Sciences and were also commercially available from a supermarket in Japan.

### Traits of New Fungal Strain “FINC-1”

The characteristics of the present plant variety will now be described in details, with relevant comparisons with varieties “TNN-11” and “Hakata-A264”.

### Juxtapositional Culture

Juxtapositional cultivation experiments were performed several times, by placing the control varieties, “FINC-1” strain and the two parental cultivars in juxtaposition with each other, for observing the reluctant reaction caused when the fungal strains were extraneous to each other. As a result, the juxtapositional cultivation experiments have shown that “FINC-1” was extraneous to all the parental cultivars (see Table 1A and Table 1B below, +: inhibition zone, −: no inhibition zone formation).

TABLE 1A
Results of juxtapositional cultivation experiments

| | Hokuto Shiro | Hakata- |
|---|---|---|
| Similar variety | Ichigoukin | A264 |
| Hokuto Shiro Ichigoukin | − | + |
| Hakata-A264 | + | − |

TABLE 1B
Results of juxtapositional cultivation experiments

| Similar variety | TNN-11 | Hakata-A264 |
|---|---|---|
| FINC-1 | + | + |

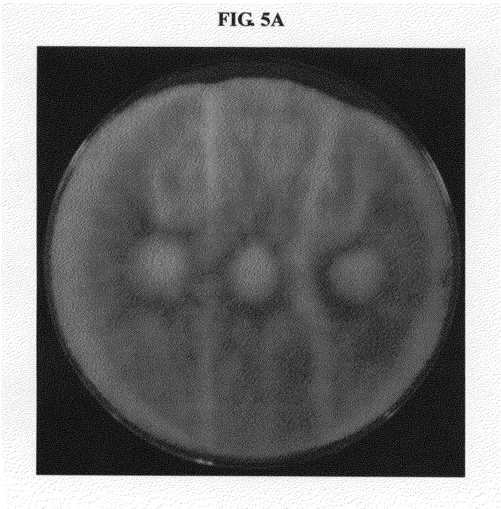

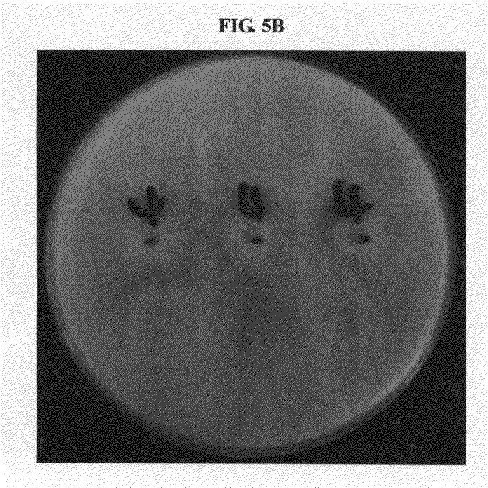

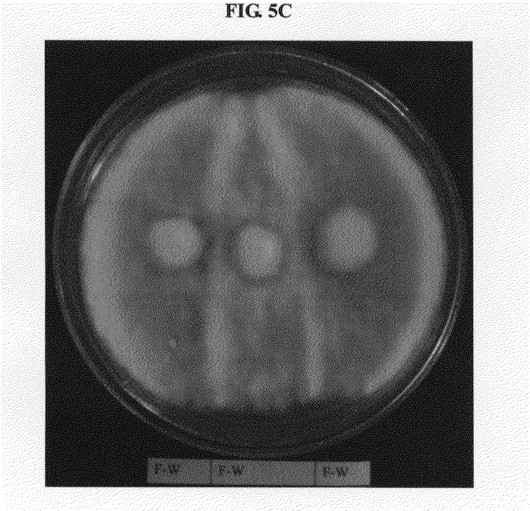

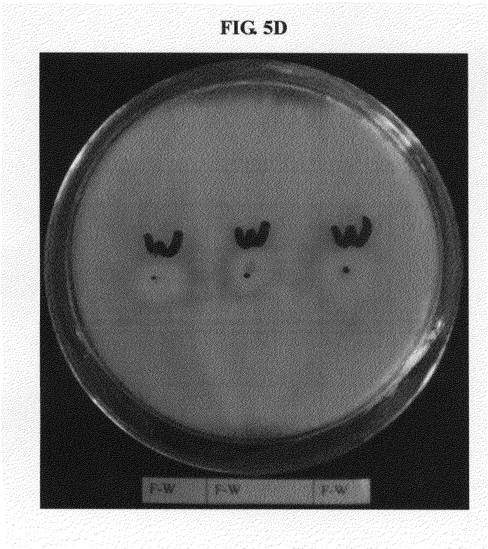

The control varieties, “Hokuto Shiro Ichigoukin” and “Hakata-A264” (well-known different cultivars), were commercially available from a supermarket in Japan. No inhibition zone was formed between the control varieties themselves and clear inhibition zone was formed between the control varieties (see Table 1A and FIG. 5A-FIG. 5F).

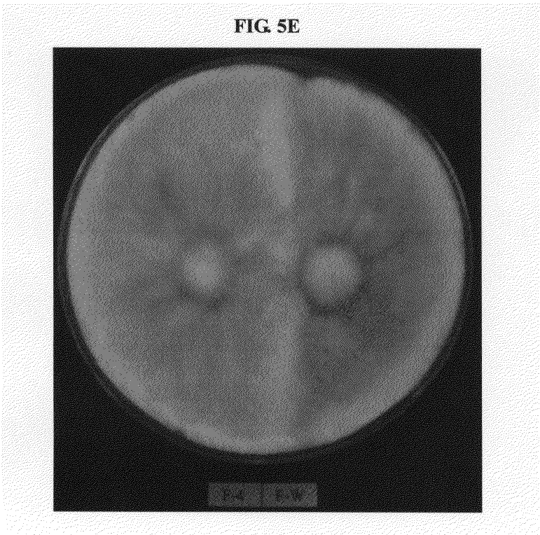

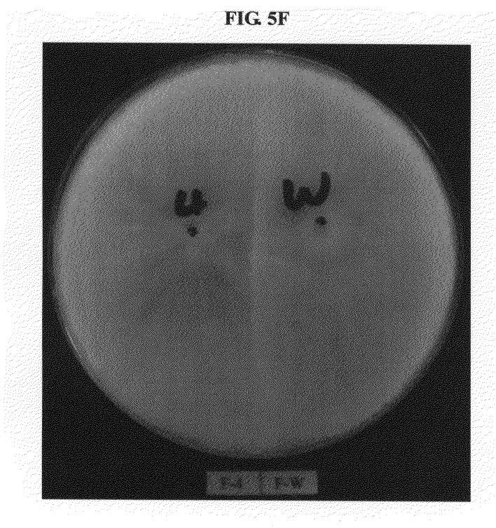

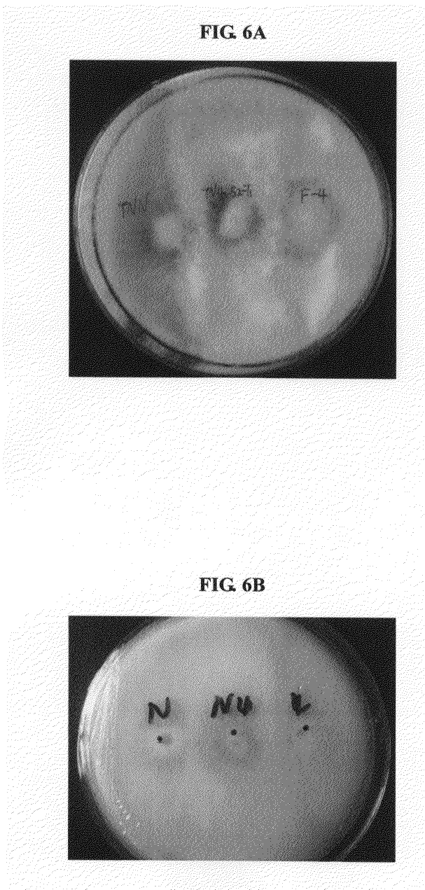

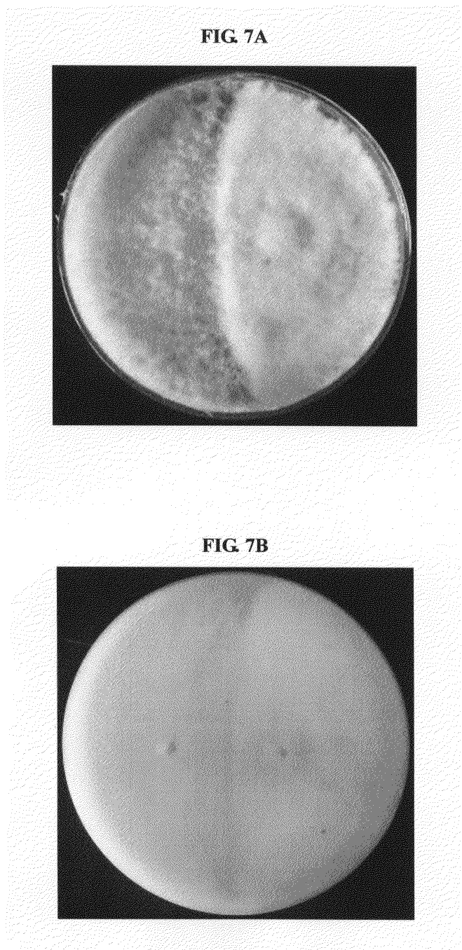

The experiments for the aforementioned reluctant reaction were performed by inoculating the “FINC-1” strains and the control strains on a potato dextrose agar medium with a distance of 3 centimeters. After conducting the cultivation at 24° C. for 26 days, the reluctant reaction of the testing strains was measured. Under the experimental condition, the inhibition zone between “FINC-1” and “TNN-11” was not as clear as the inhibition zone between “FINC-1” and Hakata-A264 (see FIG. 6A and FIG. 6B below). Juxtapositional cultivation experiments between “FINC-1” and “TNN-11” was performed once more, and clear inhibition zone was formed after conducting the cultivation at 24° C. for 35 days (see FIG. 7A and FIG. 7B below).

Length of culture from spawning to fruit-body-formation promotion, length of fruit-body-formation promotion to harvest and yield.

The trait of growth length and yield changed with the length of culture from spawning to fruit-body-formation promotion was investigated. Seven lengths of culture were established at 70, 75, 80, 85, 90, 95 and 100 days for the control “TNN-11”. The results were summarized in Table 2A below. At the same time, six lengths of culture were established at 70, 75, 80, 85, 90 and 95 days for “FINC-1”, and the results were summarized in Table 2A below.

TABLE 2A
TNN-11

| Culture | Growth | Yield |
|---|---|---|
| length (days) | length (days) | (g/bottle) |
| 70 | 27 | 135 |
| 75 | 24 | 132 |
| 80 | 25 | 146 |
| 85 | 24 | 153 |
| 90 | 24 | 157 |
| 95 | 24.5 | 171 |
| 100 | 23.5 | 160 |

TABLE 2B
FINC-1

| Culture | Growth | Yield |
|---|---|---|
| length (days) | length (days) | (g/bottle) |
| 70 | 24 | 139 |
| 75 | 23 | 171 |
| 80 | 23 | 168 |
| 85 | 21.5 | 189 |
| 90 | 21 | 185 |
| 95 | 20 | 182 |

Based on the results shown in Table 2A and Table 2B, the best length of culture of “TNN-11” and “FINC-1” were 95 days and 85 days respectively, because the yield was the highest for this length. Meanwhile, the growth lengths of “TNN-11” and “FINC-1” were 24.5 days and 21.5 days at their highest yield, respectively.

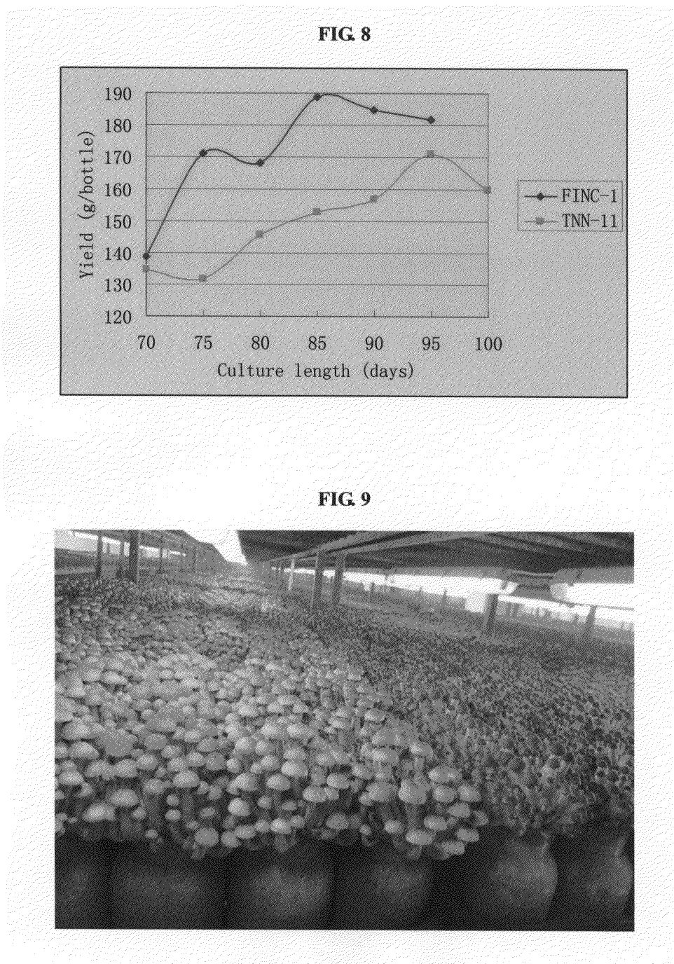

Based on the results shown in Table 2A and Table 2B, the growth lengths of “TNN-11” go up and down around 24 days under the condition of culture lengths last from 75 to 100 days, while the growth lengths of “FINC-1” go down from 23 to 20 days stably. Table 2A and Table 2B also show that the yield of “FINC-1” is higher than the yield of “TNN-11” at every growth length from 70 to 95 days, and that can be seen in FIG. 8.

Cultivation experiments were repeatedly carried out from November 2009 to August 2010 for corroborating stability, reproducibility and uniformity of the new strains under a small-scale number (2,300 bottles each time), and the results is shown in Table 3.

Based on the results shown in Table 3, “FINC-1” harvested its higher yield with its shorter culture length and growth length compared to that of “TNN-11”.

TABLE 3

| | Strain | Culture | Growth | Yield |
|---|---|---|---|---|
| | name | length (days) | length (days) | (g/bottle) |
| Exp. 1 | FINC-1 | 84 | 22 | 188 |
| | TNN-11 | 91 | 23.5 | 166 |
| Exp. 2 | FINC-1 | 85 | 22 | 177 |
| | TNN-11 | 96 | 23 | 163 |

Large-scale cultivation experiments (60,000 bottles each time) were repeatedly carried out from November 2010 to December 2011 for corroborating adaptability, and one of the results is shown in Table 4.

The experiments were carried out just as the tradition production process except for the seed fungi. The culture length was 84 days, the growth length was 21 days and the yield was 185 g/bottle, all the results were better than that of the control “TNN-11”.

TABLE 4

| | Culture | Growth | Yield |
|---|---|---|---|
| Strain name | length (days) | length (days) | (g/bottle) |
| FINC-1 | 84 | 21 | 185 |
| TNN-11 | 92 | 23 | 170 |

### Summary of Cultivation Experiments

Cultivation experiments were repeatedly carried out, which included mini-scale, small-scale and large-scale, and last from August 2008 to December 2011. All the results proved that the new variety has advantages of shorter culture length, shorter growth length, higher yield, good appearance, and it is exquisite in stability, reproducibility and uniformity when being produced.

### Eating-Quality Characteristics

First, fresh beech mushroom samples each having a fruit body of 200 grams were fried with soybean oil for 1 minute, and then, cooled at room temperature. Thereafter, blind tasting tests were conducted among 19 tasting testers to confirm the taste of the new variety “FINC-1” compared to the control varieties with respect to bitterness and firmness. The result of the tasting tests is shown in Table 5. As is evident from the tasting result, the new variety “FINC-1” was assessed by almost all of tasting testers as not bitter or slightly bitter.

Out of the 19 tasting testers, 89% of the testers appraised “FINC-1” as not bitter or slightly bitter, 68% of the testers appraised “FINC-1” as crunchy. The tasting results show that the new variety “FINC-1” is deliciously flavored.

TABLE 5

| | Numbers of testers who made valuations of the varieties | | | | |
|---|---|---|---|---|---|
| | bitterness | | | | |
| | | Slightly | not | firmness | |
| Strain name | bitter | bitter | bitter | Crunchy | Not crunchy |
| FINC-1 | 2 | 5 | 12 | 13 | 6 |
| TNN-11 | 1 | 4 | 14 | 9 | 10 |
| Hakata-A264 | 16 | 3 | 0 | 14 | 5 |

### Morphological Characteristics of “FINC-1” Cultivated Experimentally

The cultivation was conducted using bottles having a capacity of 850 cc and containing sawdust culture medium. The culture medium mainly contain mixed wood sawdust of broad leaf tree with a little corn meal, rice bran and wheat bran and so on. After cultivated at 22° C. for 85 days fungus scratching and sprouting of seed fungi were performed at a temperature of 14° C. Thereafter, the fungal strains thus bred were further nurtured at a temperature of 15° C., and at a changed illumination intensity based on the growth state. The air humidity was at a humidity of 95% from fruit-body-formation promotion to harvest. Finally, the grown mushrooms were harvested when the caps thereof reach maturity.

The new variety “FINC-1” grown in the manner as described above has the following trait characteristics. The “FINC-1” has a dark brown cap until one or two days before harvested (see FIG. 1C), but it changed to a yellow tea color cap when the caps finally reach maturity (see FIG. 1D and FIG. 9). Distinct rounded mottles are distributed all over the cap when the cap is young (see FIG. 1C), and the ratio of cap covered by the distinct mottles become smaller little by little with the cap growth, then only the upper surface of the fungous pileus (except for the periphery) was covered by the mottles (see FIG. 1B). The gills of the fungus are greyish white and normal. The stem is greyish white, higher, centric to the cap, not hairy and medium-sized diameter.

As is apparent from the foregoing description, the new variety “FINC-1” has advantages of short culture length, short growth length, high yield, good appearance, no bitterness, and it is exquisite in stability, reproducibility and uniformity when being produced. “FINC-1” expressly is distinct in its genotype and morphological trait character, which make it a distinction from the existing varieties of this sort.

What is claimed is:

1. A new and distinct variety of beech mushroom plant as substantially illustrated or described in the specification.
